(19) United States
(12) Patent Application Publication
(10) Pub. No.: US 2022/0213204 A1
(43) Pub. Date: Jul. 7, 2022

(54) CD25-SPECIFIC CHIMERIC ANTIGEN RECEPTORS AND THEIR USES

(71) Applicants: UNIVERSITÄT REGENSBURG, REGENSBURG (DE); UNIVERSITÄT ZU KÖLN MEDIZINISCHE FAKULTÄT, KÖLN (DE)

(72) Inventors: HINRICH ABKEN, GEISELHOERING-HAINSBACH (DE); ANDREAS HOMBACH, BRÜHL (DE); MANUEL EHLING, OCHTRUP (DE)

(21) Appl. No.: 17/613,218

(22) PCT Filed: Jun. 3, 2020
(86) PCT No.: PCT/EP2020/065368

§ 371 Date: Nov. 22, 2021

(30) Foreign Application Priority Data
Jun. 3, 2019 (EP) 19177859.6

(51) Int. Cl. C07K 16/28; A61K 35/17; A61P 37/00; C07K 14/725; C07K 14/705; C07K 14/715; C12N 5/0783

(52) U.S. Cl. CPC C07K 16/2866; A61K 35/17; A61P 37/00; A61K 2039/5156; C07K 14/70521; C07K 14/7155; C12N 5/0636; C07K 14/7051

(57) ABSTRACT

The present invention relates to proteins which comprise (i) a CD25-specific binding domain, (ii) a linker domain, connecting domain (i) and domain (iii), (iii) a transmembrane domain, and (iv) a signalling domain. The present invention furthermore relates to nucleic acids encoding the proteins, expression constructs for expressing the protein in a host cell and host cells. The present invention further relates to pharmaceutical compositions comprising said protein(s), nucleic acid(s), expression construct(s) or host cell(s). The proteins of the invention are CD25-specific chimeric antigen receptors that are suitable for generating CD25-specific immune cells, which can be used e.g. in the treatment of inflammation.

The present invention relates to proteins which comprise (i) a CD25-specific binding domain, (ii) a linker domain, connecting domain (i) and domain (iii), (iii) a transmembrane domain, and (iv) a signalling domain. The present invention furthermore relates to nucleic acids encoding the proteins, expression constructs for expressing the protein in a host cell and host cells. The present invention further relates to pharmaceutical compositions comprising said protein(s), nucleic acid(s), expression construct(s) or host cell(s). The proteins of the invention are CD25-specific chimeric antigen receptors that are suitable for generating CD25-specific immune cells, which can be used e.g. in the treatment of inflammation.

### BACKGROUND OF THE INVENTION

Auto-immune reaction has been found to be an underlying cause in many diseases; chronic inflammation is the main consequence of an auto-immune reaction and occurs when the auto-immune reaction is not limited and out of control. Numerous diseases are associated with chronic inflammation with a lasting and de-regulated activation of the cellular immune response. More than 80 auto-immune diseases exist; these are, for instance, multiple sclerosis, inflammatory bowel disease including Crohn's disease and ulcerative colitis, type-1 diabetes, psoriasis, rheumatoid arthritis, systemic lupus erytematosus, Hashimoto's thyreoiditis, Addison's disease, Graves' disease, Sjögren's syndrome, myastenia gravis, auto-immune vasculitis, pernicious anemia, and celiac disease.

Regulatory T cells (Treg cells) inhibit sustained inflammatory reactions, which is, however, not very effective in case of auto-immune diseases; the inflammatory reaction continues despite the presence of Treg cells. Steroids are regularly used for a therapeutic reduction of chronic inflammatory reactions. They, however, inhibit the inflammation only in an insufficient manner and have severe systemic effects.

Conventional treatment of chronic inflammatory diseases includes nonsteroidal anti-inflammatory drugs and immunosuppressing drugs like prednisone or cortisols that slow the immune cell attack and reduce inflammation; relapses are common after the drug is discontinued. In some auto-immune diseases such as Hashimoto's thyreoditis immunosuppressive drugs are usually not prescribed because the side effects outweight the benefits.

Thus, there is a need to inhibit deregulated inflammatory immune response at the site of inflammation in a targeted and lasting manner.

### SUMMARY OF THE INVENTION

According to the present invention this object is solved by a protein comprising
(i) a CD25-specific binding domain, preferably comprising an anti-CD25 single chain Fv (scFv) fragment;
(ii) a linker domain, connecting domain (i) and domain (iii), preferably comprising a human immunoglobulin Fc domain, more preferably comprising human IgG1 Fc;
(iii) a transmembrane domain, and
(iv) a signalling domain,
   wherein the signalling domain comprises
   a primary human signalling chain,
      preferably derived from human CD3 zeta chain or human FcεRI gamma chain,
   an intracellular co-stimulatory signalling chain,
      preferably derived from human CD28, 4-1BB, OX40 or CD27,
   or
   a fusion of said intracellular co-stimulatory signal chain(s) with the intracellular domain of a primary human signalling chain.

The protein of the present invention is a chimeric antigen receptor (CAR).

According to the present invention this object is furthermore solved by a nucleic acid encoding the CAR.

According to the present invention this object is furthermore solved by an expression construct for expressing the CAR.

According to the present invention this object is furthermore solved by a host cell expressing the CAR or comprising the nucleic acid or the expression construct.

According to the present invention this object is furthermore solved by a pharmaceutical composition, comprising
(i) at least one protein, nucleic acid, expression construct or host cell of the present invention, and
(ii) optionally, pharmaceutically acceptable excipient(s) and/or carrier, preferably harbouring (i).

According to the present invention this object is furthermore solved by using the CAR protein, nucleic acid, or expression construct for generating CD25-specific immune cells.

According to the present invention this object is furthermore solved by the CAR protein, nucleic acid, expression construct or host cell for use as a medicament.

According to the present invention this object is furthermore solved by the CAR protein, nucleic acid, expression construct or host cell for use in the treatment of inflammation.

According to the present invention this object is furthermore solved by the CAR protein, nucleic acid, expression construct or host cell for use in target-cell specific immunotherapy.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to 21” should be interpreted to include not only the explicitly recited values of 1 to 21, but also include individual values and sub-ranges within the indicated range. Thus, included in this numerical range are individual values such as 1, 2, 3, 4, 5 . . . 17, 18, 19, 20, 21 and sub-ranges such as from 2 to 10, 8 to 15, etc. This same principle applies to ranges reciting only one numerical value, such as “at least 90%”. Furthermore, such an interpretation should apply regardless of the breadth of the range or the characteristics being described.

### CD25-Specific CARs

As outlined above, the present invention provides CD25-specific chimeric antigen receptors (CARs).

The present invention provides a multi-domain or modular protein comprising
(i) a CD25-specific binding domain,
(ii) a linker domain, connecting domain (i) and domain (iii),
(iii) a transmembrane domain,
and
(iv) a signalling domain,
   wherein the signalling domain comprises
   a primary human signalling chain,
      preferably derived from human CD3 zeta chain or human FcεRI gamma chain,
   an intracellular co-stimulatory signalling chain,
      preferably derived from human CD28, 4-1BB, OX40 or CD27,
   or
   a fusion of said intracellular co-stimulatory signal chain(s) with the intracellular domain of a primary human signalling chain; and,
optionally, further comprising a secretion signal peptide.

In one embodiment, said domain (i) is a monospecific domain, i.e. it is only specific for CD25.

The proteins of the invention are preferably cell surface receptor proteins and, thus, comprise an extracellular portion (domains (i) and (ii)), a transmembrane portion (domain (iii)) and a cytoplasmic portion (domain (iv)), and can thus be inserted into the outer cell membrane of the host cell. The functionality of the proteins of the invention within a host cell is detectable in an assay suitable for demonstrating the signaling potential of said protein upon binding of a particular ligand. Such assays are available to the skilled artisan.

Upon binding to the CD25, i.e. the target, such chimeric antigen receptors link to endogenous signalling pathways in a cell (an immune cell) and generate certain activating signals (depending on the signalling domain).

### (i) CD25-Specific Binding Domain

CD25 is the α-chain of the interleukin 2 (IL-2) receptor, and when expressed with α- and β-chain, the receptor acquires high affinity for IL-2. CD25 is expressed on the surface of immune cells, preferably on activated immune cells.

The binding domain serves for the targeting of the protein according to the present invention or a respective cell expressing/carrying the protein according to the present invention on its surface to a target cell carrying CD25 on its surface. In a preferred embodiment, said binding domain is monospecific for CD25.

Binding of the binding domain of the CAR to its cognate CD25 target on the surface of target cells furthermore results in transmitting a signal into the CAR-expressing immune cells via the intracellular signalling domain(s) of the CAR protein which activates the immune cell to execute a variety of effector functions including amplification, cytokine release, target cell elimination or repression and others.

A (target) binding domain of a CAR is usually derived from an antigen binding domain derived from an antibody against an antigen or receptor of the target, or a peptide binding an antigen or receptor of the target, or a peptide or protein ligand binding a receptor on the target.

In an embodiment, where domain (i) is derived from an antigen binding domain, the antigen binding domain is preferably derived from an antibody or an antibody fragment, such as a single chain Fv (scFv) fragment, a Fab fragment, a diabody, a variable domain of the antibody heavy chain or antibody light chain, a DARPin, an anticalin or any peptide or protein with specificity in binding to CD25.

In one embodiment, the extracellular CD25-specific binding domain (i) is derived from or comprises or consists of IL-2.

In a preferred embodiment, the CD25-specific binding domain (i) comprises or consists of an anti-CD25 single chain Fv (scFv) fragment.

In one embodiment, CD25-specific binding domain (i) comprises or consists of the amino acid sequence of SEQ ID NO. 1 [=amino acid sequence of the anti-CD25 scFv].

QVKLQQSGTVLARPGASVKMSCKASGYRFTNYWMHWVKQRPGQGLEWIGV
IYPGNSDTSYNQKFKGKAKLTAVTSASTAYMELSSLTNEDSAVYYCTREG
EGSDYWGQGTTVTVSSGGGGSGGGGSGGGGSQIVLTQSPATMAASPGEKI
TITCSASSSISSNYLHWYQQKPGFSPKLLIYRTSNLASGVPARFSGSGSG
TSYSLTIGTMEAEDVATYYCQQGSSIPYTFGGGTKLELK

### (ii) Linker Domain

The linker domain or linker region (ii) connects the binding domain (i) and the transmembrane domain (iii).

The linker region serves as a spacer between the binding domain (i) and the transmembrane domain (iii).

In a preferred embodiment, the linker domain (ii) comprises a human immunoglobulin Fc domain, more preferably comprises or consists of human IgG1 Fc domain.

The IgG1 Fc domain may comprise the hinge-CH1-CH2-CH3 domain or parts thereof.

In one embodiment, the linker domain (ii) comprises or consists of the amino acid sequence of SEQ ID NO. 2 [=amino acid sequence of the linker domain IgG1-Fc]:

DPAEPKSPDKTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPGKK

The IgG1 Fc domain may comprise the hinge-CH1-CH2-CH3 domain or parts thereof. The domain may furthermore harbour a mutation to diminish binding to the Fc receptor. The mutation is described in Hombach et al. (2010), and comprises the amino acid residues PPVA-G(232-237) ( . . . ) IAR(253-255), see SEQ ID NO. 3 below, instead of the amino acid residues PELLGG(232-237) ( . . . ) ISR(253-255), see SEQ ID NO. 2 above.

In this embodiment, the linker domain (ii) comprises or consists of the amino acid sequence of SEQ ID NO. 3 [=amino acid sequence of the linker domain IgG1-ΔFc]:

PAEPKSPDKTHTCPPCPAPPVAGPSVFLFPPKPKDTLMIARTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGKK

(iii) Transmembrane Domain

The transmembrane domain or transmembrane region anchors the protein of the present invention on the cell membrane.

The transmembrane domain is preferably derived from CD4, CD8, CD3, CD28 or 4-1BB, more preferably CD28. Any other transmembrane domain or region can likewise be used.

(iv) Signalling Domain

The signalling domain (iv) comprises one or more intracellular signalling domains.

The signalling domain (iv) is suitable for activating immune cells

The signalling domain serves the coupling of the target/antigen recognition to the intracellular signalling machinery.

Binding of the CD25-specific binding domain (i) of the CAR to its cognate target CD25 on the surface of target cells furthermore transmits a signal into the CAR-expressing immune cells via the intracellular signalling domain(s) of the CAR which activates the cell-intrinsic activity of such immune cells.

The signalling domain (iv) comprises or consists of (is)
a primary human signalling chain,
intracellular co-stimulatory signalling chain(s),
   or
a fusion of said intracellular co-stimulatory signalling chain(s) with the intracellular domain of a primary human signal chain

Said primary human signalling chain is preferably derived from
human CD3 zeta chain, or
human FcεRT gamma chain
or parts thereof.

Said intracellular co-stimulatory signalling chain(s), which can be part of the fusion, are preferably derived from human CD28, 4-1BB, OX40 or CD27, more preferably CD28.

In a preferred embodiment, the transmembrane domain (iii) is derived from the CD3ζ chain or the CD28 costimulatory molecule. Transmembrane domains from other molecules like CD4 or CD8 can likewise be used.

In a preferred embodiment, the signalling domain (iv) is derived or selected from
(1) the human CD3 zeta chain,
(2) the intracellular domain of human CD28 linked to the intracellular domain of human CD3 zeta chain, or
(3) the intracellular domain of human CD28 linked to the intracellular domain of human CD3 zeta chain, wherein CD28 comprises at least one mutation.

In a preferred embodiment, the signaling domain (iv) is or comprises or consists of the intracellular domain of human CD28 linked to the intracellular domain of human CD3 zeta chain; or it is or comprises or consists of the intracellular domain of human CD28 linked to the intracellular domain of human CD3 zeta chain, wherein CD28 comprises at least one mutation.

In a preferred embodiment, said mutation in CD28 is a deletion of the binding site for Lck (lymphocyte-specific protein kinase), such as described in SEQ ID NO. 13, which shows the CD28ΔLck intracellular domain:

RSKRSRLLHSDYMNMTPRRPGPTRKHYQAYAAARDFAAYRS

Deletion of the Lck binding site within the CD28 costimulatory domain of the CAR results in the induction of IL-2 release upon CAR signaling is substantially reduced. Other T cell effector functions initiated by the CAR are not affected like T cell amplification, IFN-g release or cytolytic activities.

In a particularly preferred embodiment, the protein (or CAR) of the present invention comprises a binding domain (i) which is monospecific for CD25, and a signaling domain (iv) which comprises a fusion of intracellular co-stimulatory signal chain(s) with the intracellular domain of a primary human signal chain.

### (v) Further Domains or Components

In one embodiment, the CAR of the present invention further comprises an N-terminal secretion signal (leader) peptide. Accordingly, in embodiments, where a CAR of the present invention comprises or consists of domains (i)-(iv), as described herein, it may still optionally additionally include a secretion signal (leader) peptide.

Said “secretion signal peptide” (or “secretion signal (leader) peptide”) refers to a peptide sequence that directs the transport of the CAR of the invention to the cell membrane and cell surface. It, thus, allows correct localization of the CAR, in particular the extracellular portion (domains (i) and (ii)) on the cell surface; the transmembrane portion (domain (iii)) inserted into the plasma membrane and the cytoplasmic portion (domain (iv)) in the host cell.

In an embodiment, the secretion signal peptide comprises or is immunoglobulin heavy chain signal peptide, or immunoglobulin light chain signal peptide, such as the IgG kappa light chain leader sequence.

An example of a suitable secretion signal peptide is the human IgG kappa light chain leader sequence having an amino acid sequence:

(SEQ ID NO: 15)
MDFQVQIFSFLLISASVIMSR,

or having a nucleotide sequence coding for such amino acid sequence.

In one embodiment, said human IgG kappa light chain leader sequence has a nucleotide sequence:

(SEQ ID NO: 16)
ATGGATTTTCAGGTGCAGATTTTCAGCTTCCTGCTAATCAGTGCCTCAGT
CATAATGTCTAGA

In one embodiment, the protein (or CAR) of the present invention comprises
(i) a binding domain which is monospecific for CD25;
(ii) a linker domain, as defined herein;
(iii) a transmembrane domain, as defined herein; and
(iv) a signalling domain which is or comprises or consists of intracellular domain of human CD28 linked to the intracellular domain of human CD3 zeta chain; or (iv) a signalling domain which is or comprises or consists of the intracellular domain of human CD28 linked to the intracellular domain of human CD3 zeta chain, wherein CD28 comprises at least one mutation, preferably a deletion of the Lck binding site.

In a preferred embodiment, the CAR comprises or consists of an amino acid sequence selected from SEQ ID NOs. 4 to 6 [=full-length sequences of CAR #1035 to 1037], optionally including an N-terminal secretion signal (leader) peptide,

or the CAR comprises or consists of an amino acid sequence that has at least 85%, preferably at least 90% or at least 95% sequence identity to an amino acid sequence of SEQ ID NOs. 4 to 6.

The amino acid sequence of SEQ ID NO. 4 refers to the amino acid sequence of a CAR with the domains:

(i)[anti-CD25 scFv (Rft5 scFv)]-(ii)[IgG1 Fc]-(iii and iv)[transmembrane and intracellular domain of the human CD3 zeta chain]

CAR # 1035
SEQ ID NO. 4
QVKLQQSGTVLARPGASVKMSCKASGYRFTNYWMHWVKQRPGQGLEWIGV
IYPGNSDTSYNQKFKGKAKLTAVTSASTAYMELSSLTNEDSAVYYCTREG
EGSDYWGQGTTVTVSSGGGGSGGGGSGGGGSQIVLTQSPATMAASPGEKI
TITCSASSSISSNYLHWYQQKPGFSPKLLIYRTSNLASGVPARFSGSGSG
TSYSLTIGTMEAEDVATYYCQQGSSIPYTFGGGTKLELKDPAEPKSPDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVF
SCSVMHEALHNHYTQKSLSLSPGKKDPKLCYLLDGILFIYGVILTALFLR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR

The amino acid sequence of SEQ ID NO. 5 refers to the amino acid sequence of a CAR with the domains:

(i)[anti-CD25 scFv (Rft5 scFv)]-(ii)[IgG1 Fc]-(iii and iv)[fusion of the transmembrane and intracellular domain of human CD28 with the intracellular domain of human CD3 zeta chain]

CAR # 1036
SEQ ID NO. 5
QVKLQQSGTVLARPGASVKMSCKASGYRFTNYWMHWVKQRPGQGLEWIGV
IYPGNSDTSYNQKFKGKAKLTAVTSASTAYMELSSLTNEDSAVYYCTREG
EGSDYWGQGTTVTVSSGGGGSGGGGSGGGGSQIVLTQSPATMAASPGEKI
TITCSASSSISSNYLHWYQQKPGFSPKLLIYRTSNLASGVPARFSGSGSG
TSYSLTIGTMEAEDVATYYCQQGSSIPYTFGGGTKLELKDPAEPKSPDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVF
SCSVMHEALHNHYTQKSLSLSPGKKDPKFWVLVVVGGVLACYSLLVTVAF
IIFWVRSKRSRLLHSDYMNMTPRRPGPTRKHYQPYAPPRDFAAYRSLRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR

The amino acid sequence of SEQ ID NO. 6 refers to the amino acid sequence of a CAR with the domains:

(i)[anti-CD25 scFv (Rft5 scFv)]-(ii)[IgG1 Fc]-(iii and iv)[fusion of the transmembrane and intracellular domain of human CD28, comprising a deletion of the Lck binding site, with the intracellular domain of human CD3 zeta chain]

CAR # 1037
SEQ ID NO. 6
QVKLQQSGTVLARPGASVKMSCKASGYRFTNYWMHWVKQRPGQGLEWIGV
IYPGNSDTSYNQKFKGKAKLTAVTSASTAYMELSSLTNEDSAVYYCTREG
EGSDYWGQGTTVTVSSGGGGSGGGGSGGGGSQIVLTQSPATMAASPGEKI
TITCSASSSISSNYLHWYQQKPGFSPKLLIYRTSNLASGVPARFSGSGSG
TSYSLTIGTMEAEDVATYYCQQGSSIPYTFGGGTKLELKDPAEPKSPDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVF
SCSVMHEALHNHYTQKSLSLSPGKKDPKFWVLVVVGGVLACYSLLVTVAF
IIFWVRSKRSRLLHSDYMNMTPRRPGPTRKHYQAYAAARDFAAYRSLRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR

In accordance with embodiments of the present invention, a CAR may optionally additionally include a suitable secretion signal (leader) peptide, preferably an N-terminal secretion signal (leader) peptide, allowing a correct localization of the CAR; as an example, a CAR comprising or consisting of an amino acid sequence selected from SEQ ID NO:4-6, may optionally additionally include such a suitable secretion signal (leader) peptide, preferably an N-terminal secretion signal (leader) peptide.

The CARs differ in their signalling domain (iv) and, thus, in their induced T cell effector functions, see e.g. FIG. 1B.

CAR #1035, comprising the human CD3 zeta chain, will preferably induce effector functions of engineered T cells including IFN-γ secretion and cytolysis of CD25+ target cells. Preferably, a cytolytic T cell engineered with the CAR #1035, comprising the human CD3 zeta chain, will recognize CD25+ target cells and as a consequence will release IFN-γ, lyse the CD25+ target cells and will amplify. CD25− cells are not recognized by CAR #1035 T cells and will not specifically induce T cell activation.

CAR #1036, comprising a fusion of the transmembrane and intracellular domain of human CD28 with the intracellular domain of human CD3 zeta chain, will preferably induce effector functions of engineered T cells including IFN-γ secretion, release of IL-2 and cytolysis of CD25+ target cells.

Preferably, a cytolytic T cell engineered with the CAR #1036, comprising a fusion of the transmembrane and intracellular domain of human CD28 with the intracellular domain of human CD3 zeta chain, will recognize CD25+ target cells and as a consequence will release IFN-γ and IL-2, lyse the CD25+ target cells and will amplify. CD25− cells are not recognized by CAR #1036 T cells and will not specifically induce T cell activation.

CAR #1037, comprising a fusion of the transmembrane and intracellular domain of human CD28 (comprising a deletion of the Lck binding site) with the intracellular domain of human CD3 zeta chain, will preferably induce effector functions of engineered T cells including IFN-γ secretion, release of very low amounts of IL-2, and cytolysis of CD25+ target cells. Preferably, a cytolytic T cell engineered with the CAR #1037, comprising a fusion of the transmembrane and intracellular domain of human CD28 (comprising a deletion of the Lck binding site) with the intracellular domain of human CD3 zeta chain, will recognize CD25+ target cells and as a consequence will release IFN-γ and very low amounts of IL-2, lyse the CD25+ target cells and will amplify. CD25− cells are not recognized by CAR #1037 T cells and will not specifically induce T cell activation.

### Nucleic Acids, Expression Constructs and Host Cells

As described above, the present invention provides nucleic acids/nucleic acid molecules/isolated nucleic acid molecules encoding the proteins of the invention.

The nucleic acids according to this invention comprise DNA (such as dsDNA, ssDNA, cDNA), RNA (such as dsRNA, ssRNA, mRNA), combinations thereof or derivatives (such as PNA) thereof.

Preferably, a nucleic acid of the invention comprises
the nucleic acid encoding for the amino acid sequence of SEQ ID NO. 1 or
the nucleic acid sequence of SEQ ID NO. 7 [=nucleotide sequence of the anti-CD25 scFv],
or their complementary sequences
or sequences that have at least 85%, preferably at least 90% or at least 95% sequence identity to the above sequences.

Preferably, a nucleic acid of the invention further comprises
the nucleic acid encoding for the amino acid sequence of SEQ ID NO. 2 or 3
or
the nucleic acid sequence of SEQ ID NO. 8 [=nucleotide sequence of the IgG1 Fc] or SEQ ID NO. 9 [=nucleotide sequence of the IgG1 ΔFc],
or their complementary sequences
or sequences that have at least 85%, preferably at least 90% or at least 95% sequence identity to the above sequences.

Preferably, a nucleic acid of the invention comprises or consists of
the nucleic acid encoding for an amino acid sequence selected from SEQ ID NOs. 4 to 6
or
a nucleic acid sequence selected from SEQ ID NOs. 10 to 12 [=nucleotide sequence encoding the CAR #1035 to 1037, each including the leader peptide]
or their complementary sequences
or sequences that have at least 85%, preferably at least 90% or at least 95% sequence identity.

Preferably, the nucleic acid sequences of the present invention are human sequences or codon-optimized for the expression in mammalian cells, preferably for the expression in human cells. Codon-optimization refers to the exchange in a sequence of interest of codons that are generally rare in highly expressed genes of a given species by codons that are generally frequent in highly expressed genes of such species, such codons encoding the same amino acids as the codons that are being exchanged.

Within the scope of this invention are also the nucleotide sequences obtained due to the degeneration of the genetic code of the above nucleotide sequences.

The nucleotide sequence of SEQ ID NO. 10 refers to the nucleotide sequence of a CAR with the domains:

[leader peptide]-(i)[anti-CD25 scFv (Rft5 scFv)]-(ii)[IgG1 Fc]-(iii and iv)[transmembrane and intracellular domain of the human CD3 zeta chain]

CAR # 1035
SEQ ID NO. 10
atggattttcaggtgcagattttcagcttcctgctaatcagtgcctcagt
cataatgtctagacaggtgaagctgcagcagtctgggactgtgctggcaa
ggcctggggcttccgtgaagatgtcctgcaaggcttctggctacaggttt
accaactactggatgcactgggtaaaacagaggcctggacagggtctaga
atggattggtgttatttatcctggaaatagtgatactagctacaaccaga
agttcaagggcaaggccaaactgactgcagtcacatccgccagcactgcc
tacatggagctcagcagcctgacaaatgaggactctgcggtctattactg
tacaagagagggagaaggctctgactactggggccaagggaccacggtca
ccgtctcctcaggtggaggcggttcaggcggaggtggctctggcggtggc
ggatcgcaaattgttctcacccagtctccagcaaccatggctgcatctcc
cggggagaagatcactatcacctgcagtgccagctcaagtataagttcca
attacttgcattggtatcagcagaagccaggattctcccctaaactcttg
atttataggacttccaatctggcttctggagtcccagctcgcttcagtgg
cagtgggtctgggacctcttactctctcacaattggcaccatggaggctg
aagatgttgccacttactactgccagcagggtagtagtataccgtacacg
ttcggaggggggaccaagctggagctgaaggatcccgccgagcccaaatc
tcctgacaaaactcacacatgcccaccgtgcccagcacctgaactcctgg
ggggaccgtcagtcttcctcttccccccaaaacccaaggacaccctcatg
atctcccggacccctgaggtcacatgcgtggtggtggacgtgagccacga
agaccctgaggtcaagttcaactggtacgtggacggcgtggaggtgcata
atgccaagacaaagccgcgggaggagcagtacaacagcacgtaccgggtg
gtcagcgtcctcaccgtcctgcaccaggactggctgaatggcaaggagta
caagtgcaaggtctccaacaaagccctcccagcccccatcgagaaaacca
tctccaaagccaaagggcagccccgagaaccacaggtgtacaccctgccc
ccatcccgggatgagctgaccaagaaccaggtcagcctgacctgcctggt
caaaggcttctatcccagcgacatcgccgtggagtgggagagcaatgggc
agccggagaacaactacaagaccacgcctcccgtgctggactccgacggc
tccttcttcctctacagcaagctcaccgtggacaagagcaggtggcagca
ggggaacgtcttctcatgctccgtgatgcatgaggctctgcacaaccact
acacgcagaagagcctctccctgtctccgggtaaaaaagatcccaaactc
tgctacctgctggatggaatcctcttcatctatggtgtcattctcactgc
cttgttcctgagagtgaagttcagcaggagcgcagacgcccccgcgtacc
agcagggccagaaccagctctataacgagctcaatctaggacgaagagag
gagtacgatgttttggacaagagacgtggccgggaccctgagatgggggg
aaagccgagaaggaagaaccctcaggaaggcctgtacaatgaactgcaga
aagataagatggcggaggcctacagtgagattgggatgaaaggcgagcgc
cggaggggcaaggggcacgatggcctttaccagggtctcagtacagccac
caaggacacctacgacgcccttcacatgcaggccctgccccctcgctaa

The nucleotide sequence of SEQ ID NO. 11 refers to the nucleotide sequence of a CAR with the domains:

[leader peptide]-(i)[anti-CD25 scFv (Rft5 scFv)]-(ii)[IgG1 Fc]-(iii and iv)[fusion of the transmembrane and intracellular domain of human CD28 with the intracellular domain of human CD3 zeta chain]

CAR # 1036
SEQ ID NO. 11
atggattttcaggtgcagattttcagcttcctgctaatcagtgcctcagt
cataatgtctagacaggtgaagctgcagcagtctgggactgtgctggcaa
ggcctggggcttccgtgaagatgtcctgcaaggcttctggctacaggttt
accaactactggatgcactgggtaaaacagaggcctggacagggtctaga
atggattggtgttatttatcctggaaatagtgatactagctacaaccaga
agttcaagggcaaggccaaactgactgcagtcacatccgccagcactgcc
tacatggagctcagcagcctgacaaatgaggactctgcggtctattactg
tacaagagagggagaaggctctgactactggggccaagggaccacggtca
ccgtctcctcaggtggaggcggttcaggcggaggtggctctggcggtggc
ggatcgcaaattgttctcacccagtctccagcaaccatggctgcatctcc
cggggagaagatcactatcacctgcagtgccagctcaagtataagttcca
attacttgcattggtatcagcagaagccaggattctcccctaaactcttg
atttataggacttccaatctggcttctggagtcccagctcgcttcagtgg
cagtgggtctgggacctcttactctctcacaattggcaccatggaggctg
aagatgttgccacttactactgccagcagggtagtagtataccgtacacg
ttcggaggggggaccaagctggagctgaaggatcccgccgagcccaaatc
tcctgacaaaactcacacatgcccaccgtgcccagcacctgaactcctgg
ggggaccgtcagtcttcctcttccccccaaaacccaaggacaccctcatg
atctcccggacccctgaggtcacatgcgtggtggtggacgtgagccacga
agaccctgaggtcaagttcaactggtacgtggacggcgtggaggtgcata
atgccaagacaaagccgcgggaggagcagtacaacagcacgtaccgggtg
gtcagcgtcctcaccgtcctgcaccaggactggctgaatggcaaggagta
caagtgcaaggtctccaacaaagccctcccagcccccatcgagaaaacca
tctccaaagccaaagggcagccccgagaaccacaggtgtacaccctgccc
ccatcccgggatgagctgaccaagaaccaggtcagcctgacctgcctggt
caaaggcttctatcccagcgacatcgccgtggagtgggagagcaatgggc
agccggagaacaactacaagaccacgcctcccgtgctggactccgacggc
tccttcttcctctacagcaagctcaccgtggacaagagcaggtggcagca
ggggaacgtcttctcatgctccgtgatgcatgaggctctgcacaaccact
acacgcagaagagcctctccctgtctccgggtaaaaaagatcccaaattt
tgggtgctggtggtggttggtggagtcctggcttgctatagcttgctagt
aacagtggcctttattattttctgggtgaggagtaagaggagcaggctcc
tgcacagtgactacatgaacatgactccccgccgccccgggcccacccgc
aagcattaccagccctatgcccccccacgcgacttcgcagcctatcgctc
cctgagagtgaagttcagcaggagcgcagacgcccccgcgtaccagcagg
gccagaaccagctctataacgagctcaatctaggacgaagagaggagtac
gatgttttggacaagagacgtggccgggaccctgagatggggggaaagcc
gagaaggaagaaccctcaggaaggcctgtacaatgaactgcagaaagata
agatggcggaggcctacagtgagattgggatgaaaggcgagcgccggagg
ggcaaggggcacgatggcctttaccagggtctcagtacagccaccaagga
cacctacgacgcccttcacatgcaggccctgccccctcgctaa

The nucleotide sequence of SEQ ID NO. 12 refers to the nucleotide sequence of a CAR with the domains:

[leader peptide]-(i)[anti-CD25 scFv (Rft5 scFv)]-(ii)[IgG1 Fc]-(iii and iv)[fusion of the transmembrane and intracellular domain of human CD28, comprising a deletion of the Lck binding site, with the intracellular domain of human CD3 zeta chain]

CAR # 1037
SEQ ID NO. 12
atggattttcaggtgcagattttcagcttcctgctaatcagtgcctcagt
cataatgtctagacaggtgaagctgcagcagtctgggactgtgctggcaa
ggcctggggcttccgtgaagatgtcctgcaaggcttctggctacaggttt
accaactactggatgcactgggtaaaacagaggcctggacagggtctaga
atggattggtgttatttatcctggaaatagtgatactagctacaaccaga
agttcaagggcaaggccaaactgactgcagtcacatccgccagcactgcc
tacatggagctcagcagcctgacaaatgaggactctgcggtctattactg
tacaagagagggagaaggctctgactactggggccaagggaccacggtca
ccgtctcctcaggtggaggcggttcaggcggaggtggctctggcggtggc
ggatcgcaaattgttctcacccagtctccagcaaccatggctgcatctcc
cggggagaagatcactatcacctgcagtgccagctcaagtataagttcca
attacttgcattggtatcagcagaagccaggattctcccctaaactcttg
atttataggacttccaatctggcttctggagtcccagctcgcttcagtgg
cagtgggtctgggacctcttactctctcacaattggcaccatggaggctg
aagatgttgccacttactactgccagcagggtagtagtataccgtacacg
ttcggaggggggaccaagctggagctgaaggatcccgccgagcccaaatc
tcctgacaaaactcacacatgcccaccgtgcccagcacctgaactcctgg
ggggaccgtcagtcttcctcttccccccaaaacccaaggacaccctcatg
atctcccggacccctgaggtcacatgcgtggtggtggacgtgagccacga
agaccctgaggtcaagttcaactggtacgtggacggcgtggaggtgcata
atgccaagacaaagccgcgggaggagcagtacaacagcacgtaccgggtg
gtcagcgtcctcaccgtcctgcaccaggactggctgaatggcaaggagta
caagtgcaaggtctccaacaaagccctcccagcccccatcgagaaaacca
tctccaaagccaaagggcagccccgagaaccacaggtgtacaccctgccc
ccatcccgggatgagctgaccaagaaccaggtcagcctgacctgcctggt
caaaggcttctatcccagcgacatcgccgtggagtgggagagcaatgggc
agccggagaacaactacaagaccacgcctcccgtgctggactccgacggc
tccttcttcctctacagcaagctcaccgtggacaagagcaggtggcagca
ggggaacgtcttctcatgctccgtgatgcatgaggctctgcacaaccact
acacgcagaagagcctctccctgtctccgggtaaaaaagatcccaaattt
tgggtgctggtggtggttggtggagtcctggcttgctatagcttgctagt
aacagtggcctttattattttctgggtgaggagtaagaggagcaggctcc
tgcacagtgactacatgaacatgactccccgccgccccgggcccacccgc
aagcattaccaggcc [symbol] [symbol] gacttcgcagcctatcgc
tccctgagagtgaagttcagcaggagcgcagacgcccccgcgtaccagca
gggccagaaccagctctataacgagctcaatctaggacgaagagaggagt
acgatgttttggacaagagacgtggccgggaccctgagatggggggaaag
ccgagaaggaagaaccctcaggaaggcctgtacaatgaactgcagaaaga
taagatggcggaggcctacagtgagattgggatgaaaggcgagcgccgga
ggggcaaggggcacgatggcctttaccagggtctcagtacagccaccaag
gacacctacgacgcccttcacatgcaggccctgccccctcgctaa

As described above, the present invention provides expression constructs for expressing the protein of the invention in a cell.

Preferably, the expression constructs further comprise promoter and terminator sequences.

An “expression or gene construct” (wherein both terms are used interchangeably throughout this specification) refers to a nucleic acid construct, usually an expression vector or plasmid that is used to introduce a specific gene or coding sequence into a target cell. Once the expression or gene construct is inside the cell, the encoded protein is produced by the cellular transcription and translation machinery. The expression or gene construct is designed to contain respective regulatory sequences that act as enhancer and promoter regions and lead to efficient transcription of the gene carried on the construct, including promoter and terminator sequences.

The skilled artisan can select further suitable components of expression or gene constructs.

An expression construct of the present invention is preferably transferred to T cells or other immune cells by γ-retroviral or lentiviral vectors. Alternatively, RNA transfer or DNA transfer by means of electroporation or other transfer methods known to the artisan are also applicable.

As described above, the present invention provides host cells which express a protein of the invention or which comprise a nucleic acid or an expression construct of the invention.

Preferably, the host cell is a cell of the immune system, more preferably T cells or regulatory T (Treg) cells; other cells like cells of the innate immune system like NK cells, macrophages, granulocytes can likewise be used as host cells.

### Uses and Medical Uses of the Proteins, Nucleic Acids, Expression Constructs and Host Cells and Pharmaceutical Compositions

As described above, the invention provides the use of the protein (the CAR), nucleic acid, or expression construct for generating CD25-specific immune cells.

Preferably, the invention provides the use of the protein, nucleic acid, or expression construct for generating CD25-specific T cells or CD25-specific regulatory T (Treg) cells.

As described above, the present invention provides pharmaceutical compositions.
(i) A pharmaceutical composition of the present invention comprises at least one protein of the present invention, at least one nucleic acid of the present invention, at least one expression construct of the present invention or at least one host cell of the present invention, and
(ii) optionally, pharmaceutically acceptable excipient(s) and/or carrier, preferably harbouring (i).

As described above, the present invention provides the protein (the CAR), the nucleic acid, the expression construct or the host cell for use as a medicament.

The invention provides the protein (the CAR), the nucleic acid, the expression construct or the host cell for use as a therapeutic product and/or pharmaceutical product.

As described above, the invention provides the protein (the CAR), the nucleic acid, the expression construct or the host cell for use in the treatment of inflammation.

An “inflammation” within the present invention refers to an immune cell response to stimulation by invading pathogens or endogenous signals such as damaged cells. Inflammation involves the recruitment and activation of a plethora of immune cells that results in tissue repair and return to homeostasis. As a result of local molecular, immunological and physiological processes, each tissue exhibits distinct mechanisms of inflammation, all involving immune cells or specific immune cell products like antibodies or cytokines. While physiological inflammation is self-limiting, under various pathological situations, inflammation results in uncontrolled immune cell activation, amplification and tissue damage. This is the case, for instance, during aging and senescence, during dysregulated neurological-immunological interactions, disturbance of cellular metabolism, interaction with the microbiome and others.

Preferably, “inflammation” comprises chronic inflammation, in particular in autoimmune diseases,

such as multiple sclerosis, inflammatory bowel disease including Crohn's disease and ulcerative colitis, type-1 diabetes, psoriasis, rheumatoid arthritis, systemic lupus erytematosus, Hashimoto's thyreoiditis, Addison's disease, Graves' disease, Sjögren's syndrome, myastenia gravis, auto-immune vasculitis, pernicious anemia, celiac disease, or others

Said treatment of inflammation preferably comprises reducing or suppressing the inflammatory reaction in tissues.

As described above, the invention provides the protein (the CAR), the nucleic acid, the expression construct or the host cell for use in target-cell specific immunotherapy.

### Methods for Treatment of Inflammation

The present invention further provides a method for the treatment of inflammation, in particular in auto-immune diseases.

Said method according to the present invention comprises
administering to a subject in a therapeutically effective amount
(a) a protein, a nucleic acid, an expression construct or a host cell, as obtained and defined herein, and
(b) optionally, respective excipient(s).

Said host cell is preferably a host for the protein, nucleic acid or expression construct according to the present invention.

Said method preferably comprises reducing or suppressing the inflammatory reaction in tissues.

The present invention further provides a method for targeting active immune cells.

Said method according to the present invention comprises
administering to a subject in a therapeutically effective amount
(a) a protein, a nucleic acid, an expression construct or a CD25-specific immune cell as obtained and defined herein, and
(b) optionally, respective excipient(s).

### PREFERRED EMBODIMENTS

The present invention provides chimeric antigen receptors (CARs) which target CD25, preferably target only CD25. T cells or Treg cells are provided with the anti-CD25 CARs. Such CAR-modified T cells contact active immune cells which carry CD25 on their surface and suppress inflammation at the inflammatory lesion.

CARs are recombinant transmembrane receptors which are assembled from different modules or domains and which are expressed in the surface of immune cells after gene transfer. Due to an antibody domain in their extracellular portion, CARs bind to defined target structures and convey through their intracellular portion an activation of the immune cell. Various CARs are described which are specific for tumor antigens and which target cytotoxic T cells against tumors. After binding the antigen, the cytolytic T cell is activated and lyses the cognate target cell.

This basic principle was adapted to a new concept in this invention. CARs for use in immune cells were designed and generated that target CD25 (the IL-2 receptor) on the surface of active immune cells. This is in contrast to current CARs that are designed to target tumor or other diseased cells.

In this application, we show anti-CD25 CARs which were transferred into cytotoxic T cells and suppressor Treg cells and expressed in these cells. The CAR-modified immune cells were used for suppressing an inflammatory reaction.

According to the invention, the anti-CD25 CAR-modified T cells eliminate the activated immune cells and, thus, stop the pro-inflammatory immune reaction. Since the CAR is not directed against a defined tissue, but activated immune cells, it can be used for the reduction of inflammation in any tissue.

The invention discloses, for the first time, the strategy of eliminating and suppressing inflammation cells via CAR-modified T cells which are specific for CD25. Applications are any inflammation of acute and chronic progression, in particular in the context of auto-immune diseases.

The following examples and drawings illustrate the present invention without, however, limiting the same thereto.

### BRIEF DESCRIPTION OF THE DRAWINGS

FIG. 1. Design of the anti-CD25 CARs and their domain structure.

A shown is the modular structure of an anti-CD25 CAR according to the present invention and specific embodiments of anti-CD25 CARs.

B, shown are three preferred embodiments of anti-CD25 CARs and their effect on T cell effector functions.

FIG. 2. Expression constructs of three anti-CD25 CARs.

A, Plasmid map of anti-CD25 CAR #1035

B, Plasmid map of anti-CD25 CAR #1036

C, Plasmid map of anti-CD25 CAR #1037

FIG. 3. Expression of the anti-CD25 CARs on human T cells.

Human T cells were retrovirally transduced with the expression constructs for anti-CD25 CAR #1035, anti-CD25 CAR #1036 and anti-CD25 CAR #1037, respectively. T cells without any CAR served as a control (d). After 48 hours, the CARs were detected by flow cytometry using a PE-conjugated anti-IgG1(Fc) antibody that recognizes the common extracellular IgG1(Fc) linker of the CARs. T cells were detected by staining with an anti-CD3 antibody.

FIG. 4. Specific activation of T cells.

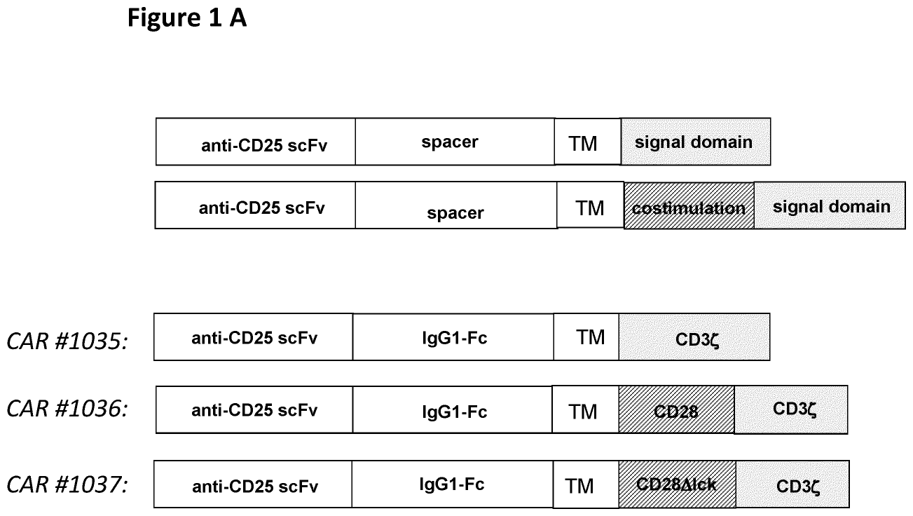

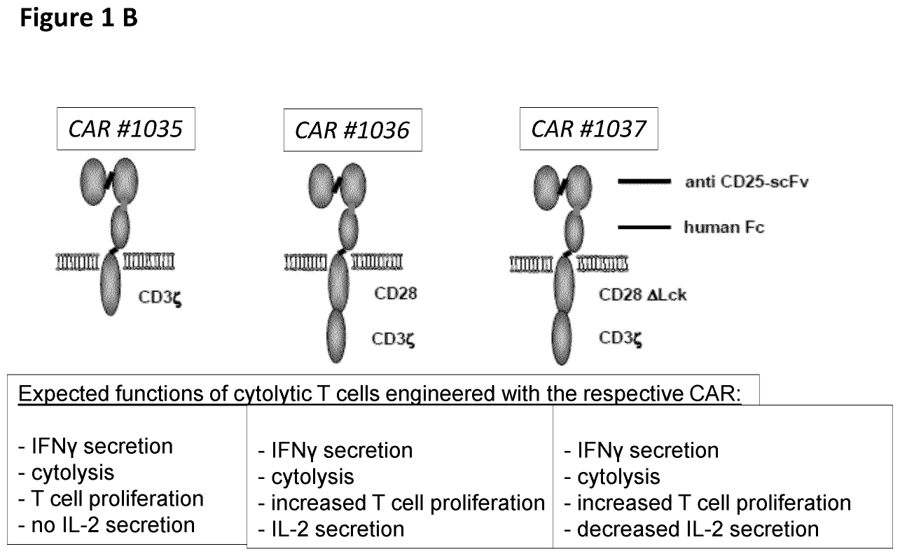

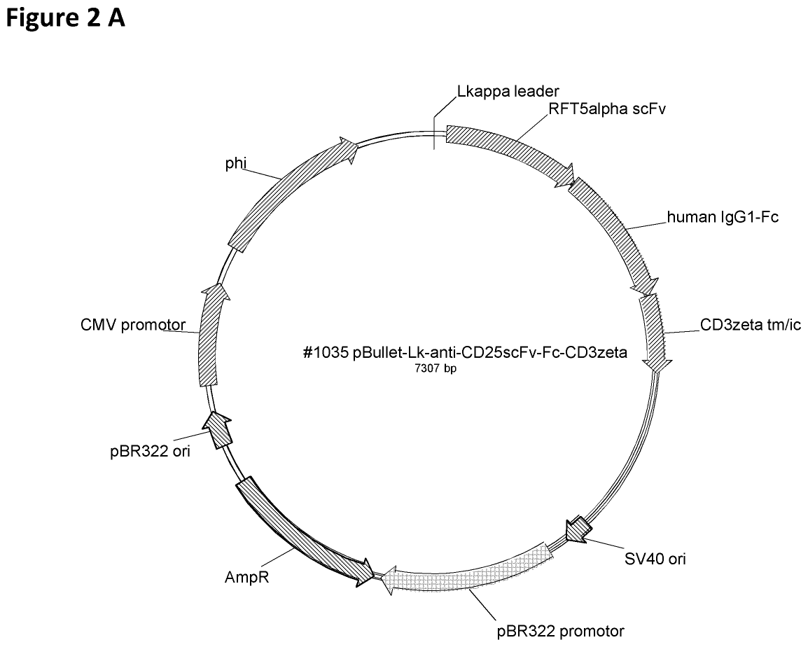

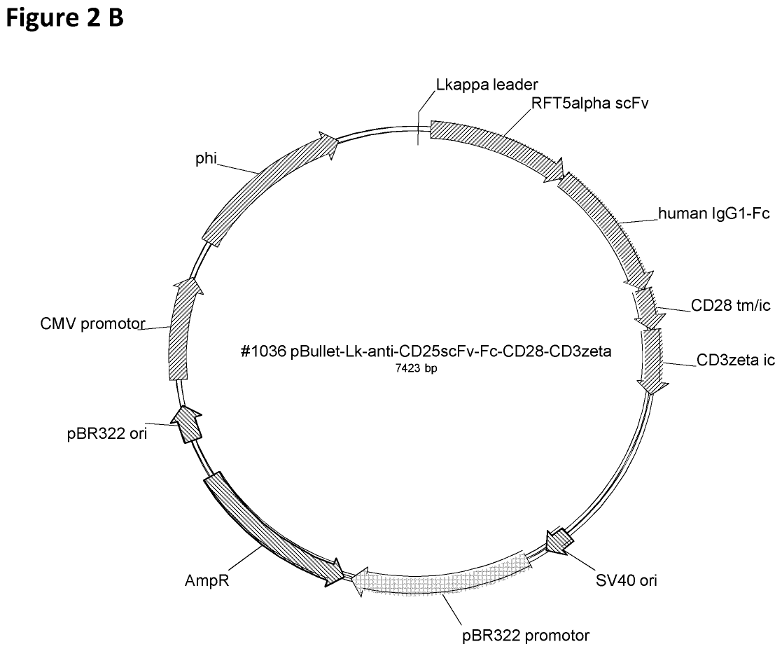

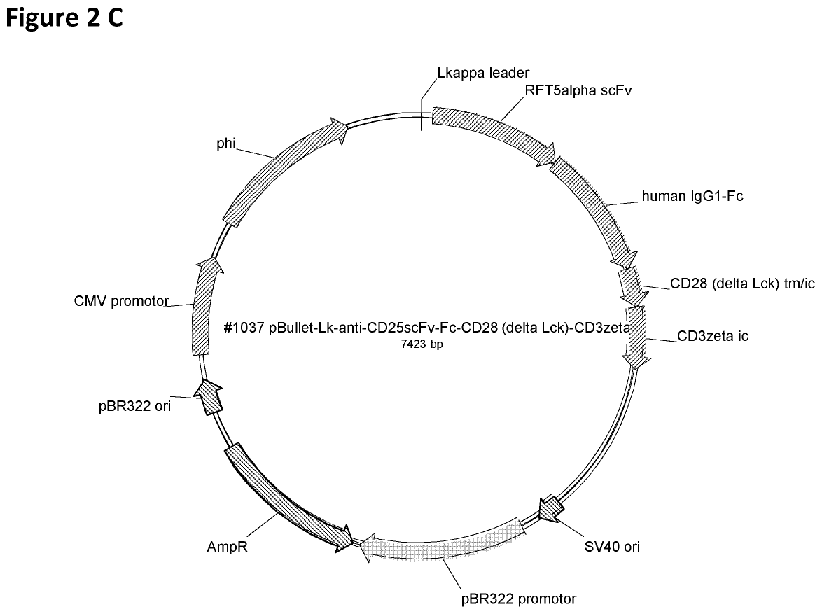

Secretion of cytokines upon CAR stimulation of CAR T cells.

Human T cells from the peripheral blood were retrovirally transduced with the expression constructs for the anti-CD25 CAR #1035, anti-CD25 CAR #1036 and anti-CD25 CAR #1037, respectively. Non-transduced T cells and T cells engineered with the anti-CEA CAR BW431/26-Fc-CD28-CD3ζ #607 served as controls. CAR T cells (10⁴ cells per well) were specifically stimulated for 48 hours by incubation on immobilized anti-human IgG1 antibody that recognizes the common extracellular IgG1 spacer. As controls, T cells were incubated with immobilized mouse IgG of irrelevant specificity (mIgG), immobilized agonistic anti-CD3 antibody OKT3, immobilized agonistic anti-CD28 antibody 15E8, or both immobilized OKT3 and 15E8, respectively. IFN-γ (a), IL-2 (b) and IL-10 in the culture supernatant were determined by ELISA. Data represent the mean±standard error of the mean.

FIG. 5. Surface expression of CD25 on CAR-modified T cells.

Human T cells were retrovirally transduced with the expression constructs for the anti-CD25 CAR #1035 (a), anti-CD25 CAR #1036 (b) and anti-CD25 CAR #1037 (c), respectively (10⁶ cells each). T cells transduced with the anti-CEA receptor BW431/26scFv-Fc-CD28-CD3ζ #607 and T cells without CAR served as controls (d and e). After 48 hours, CD25 expression by CD3⁺ T cells was detected by flow cytometry using a PE-conjugated anti-CD25 antibody and a FITC conjugated anti-CD3 antibody. CD3⁺ T cells with CD25 expression are substantially reduced in cultures with anti-CD25 CAR T cells compared with cultures with anti-CEA CAR T cells or T cells without CAR.

FIG. 6. Anti-CD25 CAR T cells reduce the cytotoxic activity of T cells with anti-CEA CAR.

T cells from the peripheral blood of the same donor were engineered with the anti-CEA CAR BW431/26-Fc-CD3ζ #700, the anti-CD25 CAR #1035 and the anti-angiomotin CAR #1061, respectively. Increasing numbers of these T cells (0.125-1×10⁴ cells per well) were incubated with CEA+ cells (10⁴ cells per well) of the LS174T line. After 48 hours, the number of living cells was determined by an XTT-based viability test; data represent the mean±standard error of the mean.

T cells with the anti-CEA CAR BW431/26-Fc-CD3ζ #700 reduce the viability of CEA+ cells of the LS174T line. When these anti-CEA CAR T cells were co-incubated with T cells with the anti-CD25 CAR #1035, the cytolytic activity against CEA+LS174T cells was reduced. For comparison, T cells expressing the anti-angiomotin CAR #1061 did not have the effect on the anti-CEA CAR #700 T cells. T cells without CAR served as further controls.

FIG. 7. Expression of CD25 on human T cells

A: Expression of CD25 T-cells without CAR

B: CD25 expression upon dose-dependent co-incubation with anti-CD25 CAR T-cells for #1035 [anti-hCD25-CD3ζ]

C: CD25 expression upon dose-dependent co-incubation with anti-CD25 CAR T-cells for #1036 [anti-hCD25-CD28-CD3ζ]

D: CD25 expression upon dose-dependent co-incubation with anti-CD25 CAR T-cells for #1037 [anti-hCD25-CD28dLck-CD3ζ]

E: CD25 expression upon dose-dependent co-incubation with anti-CD25 CAR T-cells for #1576 [anti-hCD25/anti-CEA-CD28-CD3ζ]

F: CD25 expression upon dose-dependent co-incubation with anti-CD25 CAR T-cells for #607 [anti-CEA-CD28-CD3ζ]

G: CD25 expression upon dose-dependent co-incubation with anti-CD25 CAR T-cells for #2134 [anti-mCD25-mCD4TM-mCD28-mCD3ζ] (anti-mouse CD25)

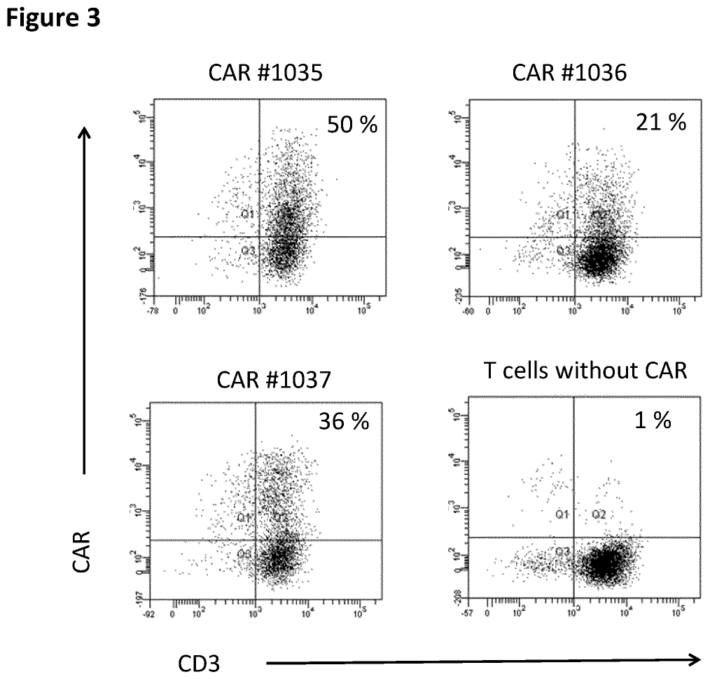

For anti-CD25 CAR T-cells, these were retrovirally transduced as described for FIG. 3.

FIG. 8. Percentage suppression of CD25 expression of co-cultivated T-lymphocytes with anti-hCD25 CAR T-cells

### EXAMPLES

### Example 1

### Materials and Methods

### 1. Cell Lines and Reagents.

The colon carcinoma cell line LS174T (ATCC CL-188) was obtained from ATCC, Rockville, Md., USA. Anti-CD3 mAb OKT3 and anti-CD28 mAb 15E8 were purified from OKT3 hybridoma (ATCC CRL 8001) and 15E8 hybridoma (kindly provided by Dr. R. van Lier, Red Cross Central Blood Bank, Amsterdam, The Netherlands) supernatants, respectively, by affinity chromatography.

Matched antibody pairs for capture and detection of human IFN-γ were purchased from BD Biosciences. Recombinant IL-2 was obtained from Endogen, Woburn, Mass., USA. Immunofluorescence was analyzed using a FACS-Canto™ cytofluorometer equipped with the Diva software (Becton Dickinson, Mountain View, Calif., USA).

### 2. Preparation of Human T Cells.

Peripheral blood lymphocytes were obtained from healthy donors by Ficoll density centrifugation. T cells were activated initially by incubation with the agonistic anti-CD3 antibody OKT3 and anti-CD28 antibody 15E8 (100 ng/ml each) and further cultivated in the presence of IL-2 (500 U/ml).

### 3. CARs.

Engineering of CARs with specificity for the carcinoembryonic antigen (CEA) and the retroviral modification of T cells was previously described in detail by Hombach et al. (2010), Weijtens et al. (1998), Hombach et al. (2001) and Hombach et al. (2000).

The generation of anti-CD25 scFv was described by Barth et al. (1998).

### 4. T Cell Modification.

Human peripheral blood T cells were retrovirally transduced for CAR expression (Golumba-Ngy et al., 2017). T cells were stimulated with OKT3 and 15E8 antibodies and transduced on day 2 or 3 by γ-retrovirus containing supernatants or by co-culture with virus producing 293T cells as described by Hombach et al. (2016). Retroviruses were produced by 293T cells upon transient transfection with the DNA of the GALV encoding and the gag/pol encoding helper plasmids, and the plasmid encoding the respective CAR. CAR expression was monitored by flow cytometry using an antibody against the common extracellular IgG1 Fc domain.

CAR expression was monitored by flow cytometry using an antibody against the common extracellular IgG1 Fc domain.

### 5. Flow Cytometry and Cell Sorting.

For flow cytometric analysis and cell sorting CAR engineered T cells were stained with fluorochrome-labeled antibodies specific for IgG1 (to detect the CAR) and CD3, respectively, and recorded by a FACSCanto II flow cytometer equipped with the FACSDiva software (BD Bioscience). CD4⁺ and CD8⁺ CAR T cells were purified by flow sorting using a FACSAria III cell sorter (BD Bioscience). Doublets were discriminated using FSC-A versus FSC-W and SSC-A versus SSC-W gating.

### 6. Statistics.

Experimental results from independent representative experiments are reported as mean values+standard deviation (SD). Significance analyzes were performed by the two-sided Student's t test using Microsoft Excel and Graphpad Prism, respectively.

### Example 2

### Expression of the Anti-CD25 CARs in Human T Cells

T cells were engineered in vitro with the anti-CD25 CAR #1035, #1036, #1037, respectively, by retroviral transduction. The CAR on the T cell surface was recorded by flow cytometry using an anti-human IgG1 antibody that recognizes the common extracellular IgG1 Fc spacer domain. T cells were recorded by staining for CD3. Transduced T cells express the CAR on the cell surface (FIG. 3). Non-transduced T cells do not express a CAR.

### Example 3

### Specific Activation of CAR T Cells by Antibody-Mediated CAR Crosslinking

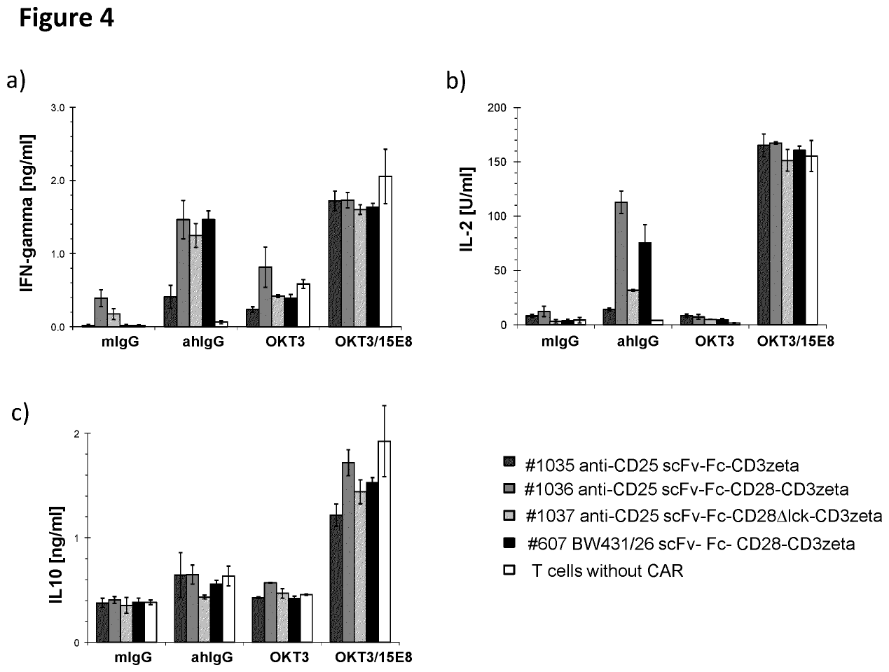

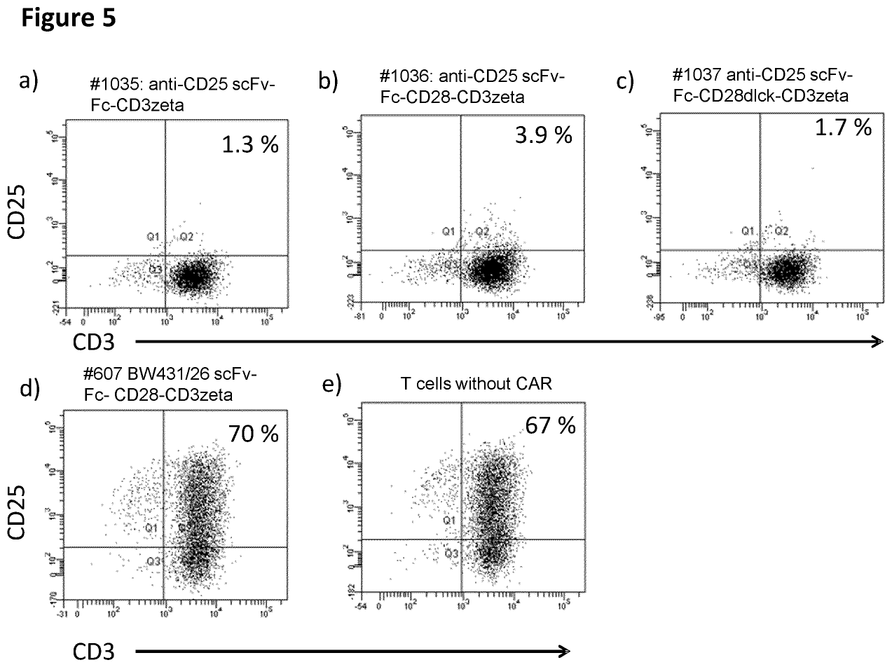

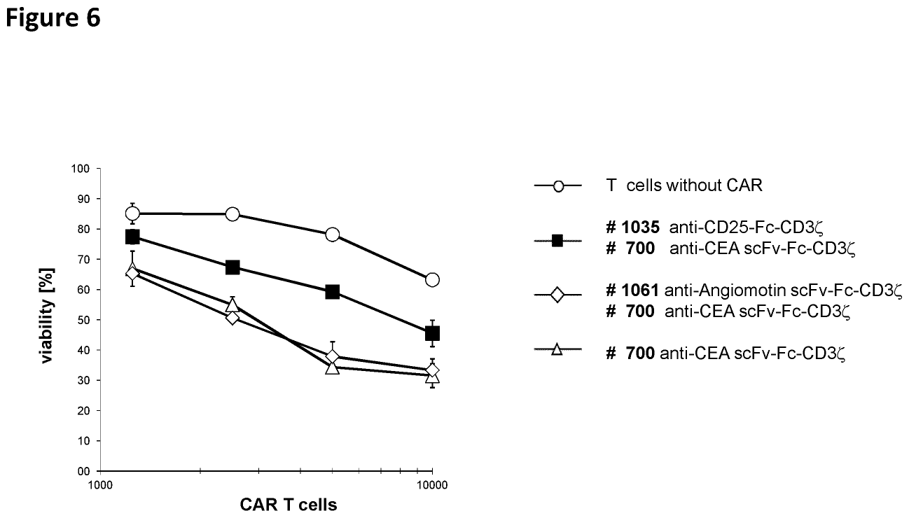

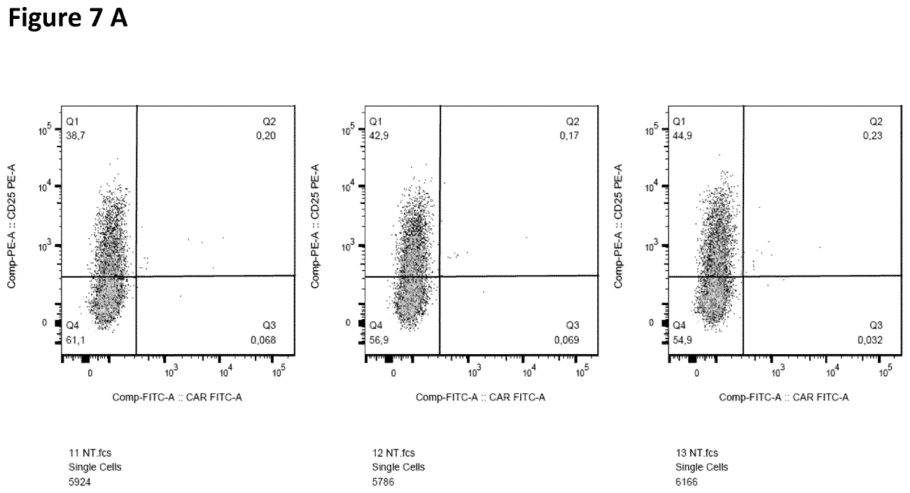

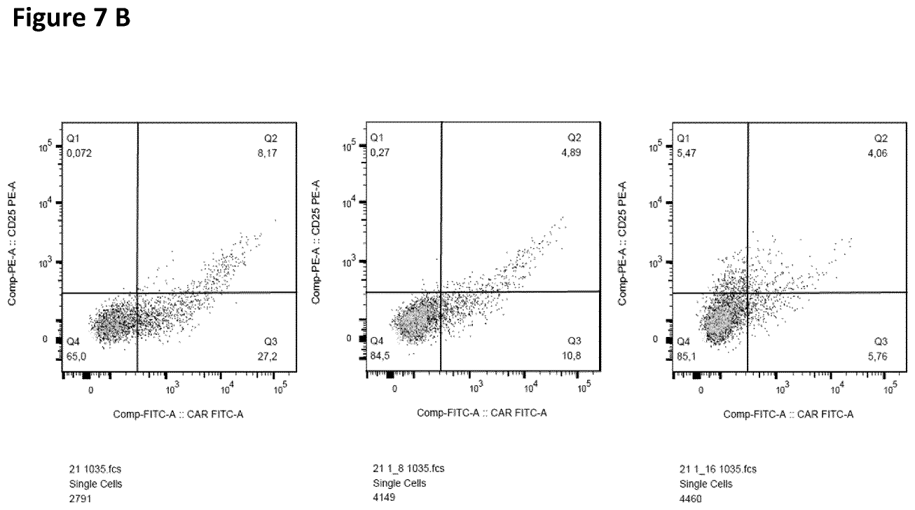

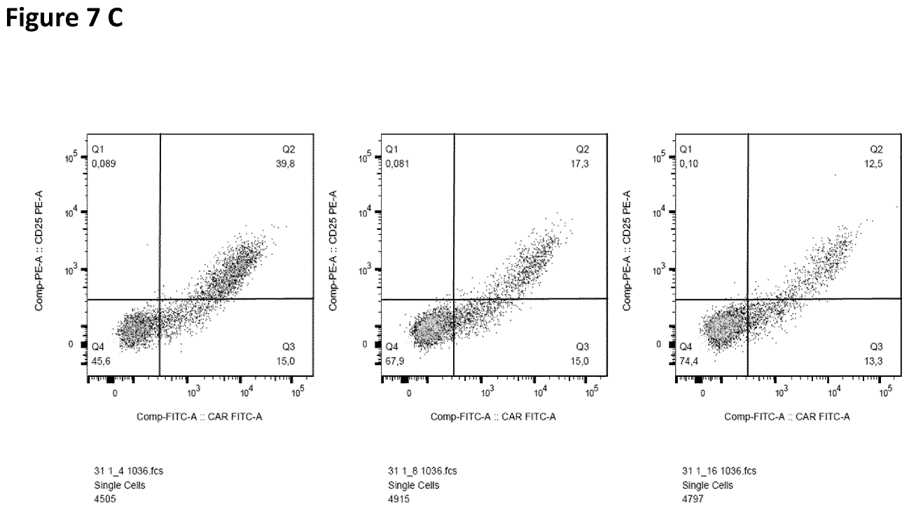

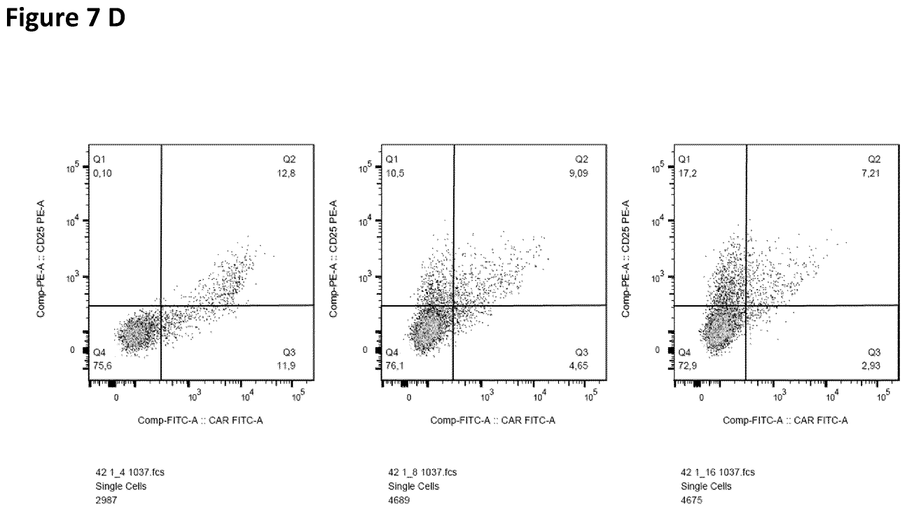

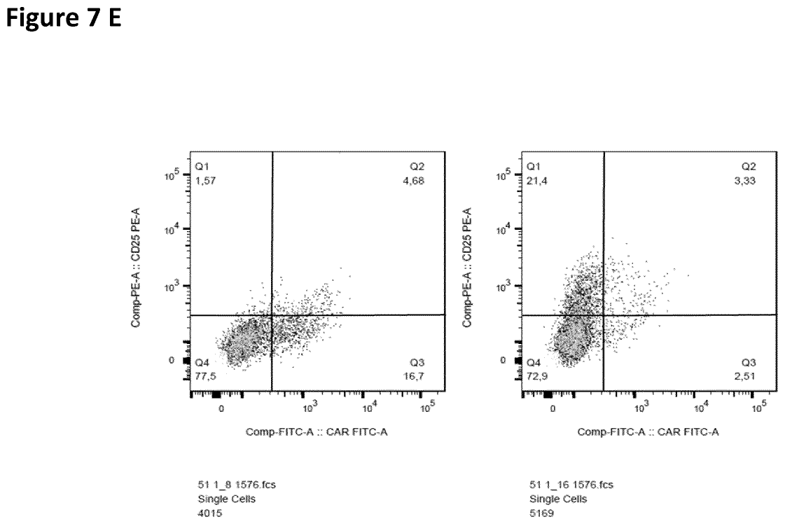

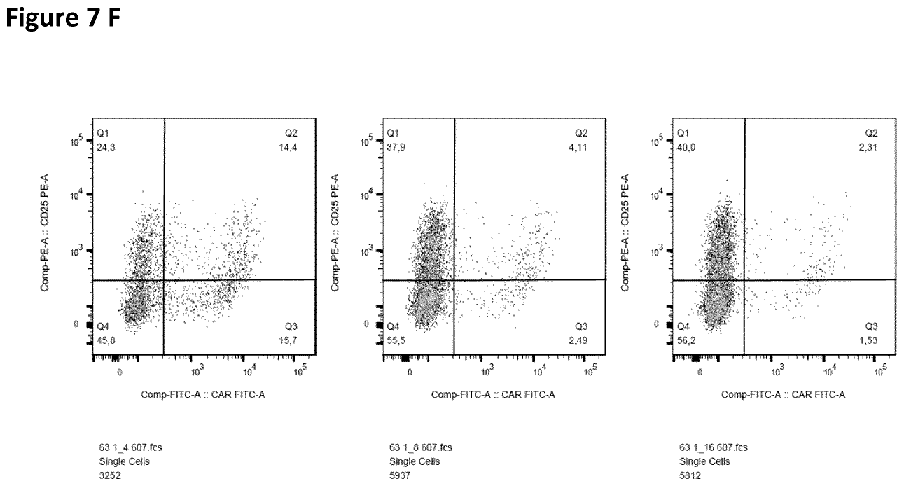

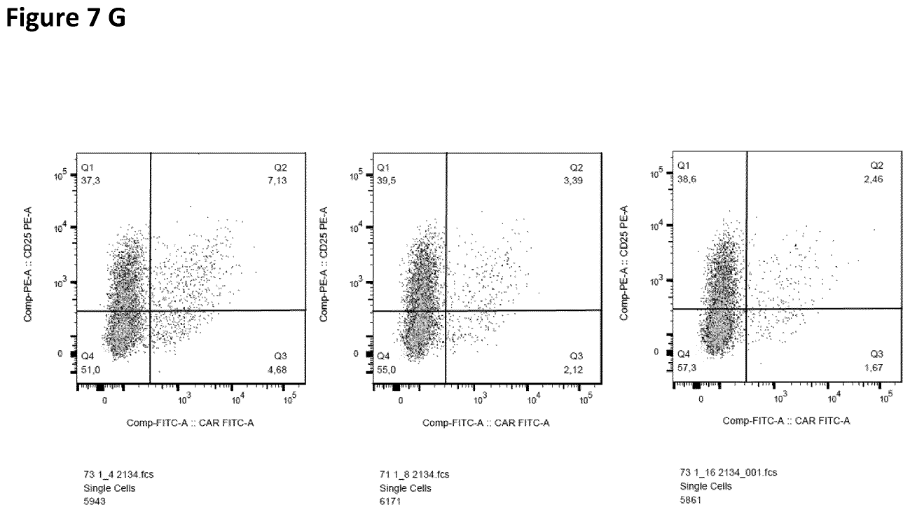

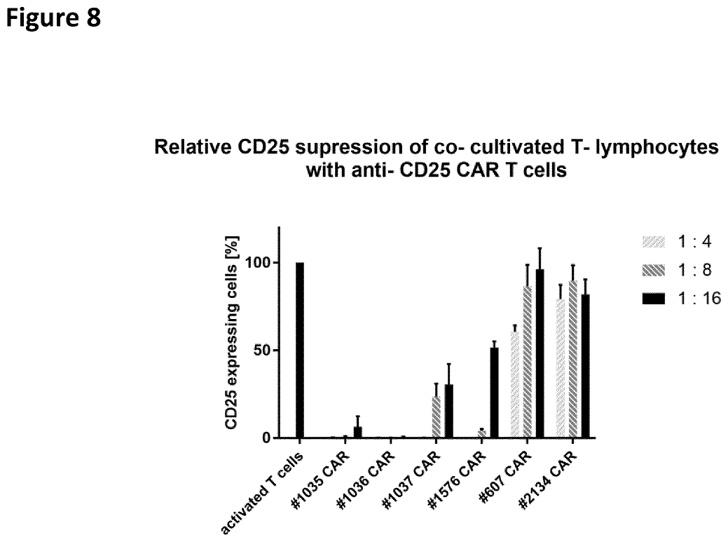

CAR T cells were assayed for CAR redirected function by antibody mediated crosslinking the CAR. Therefore, 10⁴ CAR T cells were incubated on microtiter plates coated with an anti-human IgG1 antibody that recognizes the common extracellular CAR spacer. As controls the plates were coated with a mouse IgG antibody of irrelevant specificity, with the agonistic anti-CD3 antibody OKT3, and with both the OKT3 antibody and the anti-CD28 antibody 15E8, respectively. T cells without CAR or with the anti-CEA CAR BW431/26-Fc-CD28-CD3ζ #607 served for comparison. After 48 hrs the culture supernatant was recorded for the pro-inflammatory cytokines IFN-γ and IL-2 by ELISA (FIG. 4).

Crosslinking the CAR by the immobilized anti-human IgG1 antibody induced the release of IFN-γ indicating T cell activation. Activation was specifically induced by the CAR since T cells without CAR did not increase IFN-γ release. IFN-γ levels were higher upon CD28-C CAR #1036 stimulation compared with stimulation of the CAR #1035 without CD28 costimulation. For comparison T cell activation was also obtained independently of the CAR upon TCR/CD28 stimulation after OKT3 plus 15E8 antibody incubation but less upon incubation with only OKT3.

T cells with the anti-CD25scFv-Fc-CD28-CD3ζ CAR #1036 released in addition IL-2 which was also the case for T cells with the anti-CEA CAR BW431/26scFv-Fc-CD28-CD3ζ #607. T cells with the anti-CD25 CAR without the CD28 costimulatory domain (#1035) did release only low levels of IL-2 as did T cells with the anti-CD25 CAR 1037 with CD28Δlck domain. All T cells used in the assay can equally be activated since CD3/CD28 stimulation by the antibodies OKT3 plus 15E8 produced IFN-γ release at similar levels.

### Example 4

### Anti-CD25 CAR T Cells Reduce the Number of CD25+ T Cells In Vitro

CD25 is highly expressed by activated T cells and regulatory T cells. We asked whether cytolytic T cells engineered with an anti-CD25 CAR reduce the number of CD25+ T cells in vitro.

T cells from the peripheral blood were engineered with the anti-CD25 CAR #1035, #1036, and #1037, respectively. T cells engineering with the CEA-specific CAR #700 and T cells without CAR served as controls. Expression of the respective CAR was confirmed by flow cytometry. CAR T cells were stimulated for 48 hrs with IL-2 plus the agonistic anti-CD3 antibody OKT3 and afterwards incubated without stimulation for 12 hrs until recording the number of CD25+ cells by flow cytometry.

The number of CD25+ T cells was reduced in anti-CD25 CAR T cell cultures compared with cultures that contain T cells with the anti-CEA CAR or T cells without a CAR (FIG. 5). Reduction of CD25+ T cell numbers was observed in all cultures with anti-CD25 CAR T cells indicating that each anti-CD25 CAR is capable to redirect T cells for reducing the number of CD25+ T cells.

### Example 5

### Modulating the Cytotoxicity of Anti-CEA CAR T Cells by Anti-CD25 CAR T Cells.

Anti-CEA CAR T cells produce a pro-inflammatory reaction by releasing cytokines and eliminating CEA+ cells. Anti-CD25 CAR T cells were used to repress the pro-inflammatory reaction of anti-CEA CAR T cells that serve as model for any inflammatory immune response.

Peripheral blood T cells were in vitro engineered with the anti-CEA CAR BW431/26scFv-Fc-CD3ζ #700 to be used as pro-inflammatory cells redirected against CEA+ target cells. A second population of T cells from the same donor was engineered in vitro with the anti-CD25scFv-Fc-CD3ζ CAR #1035 to serve as anti-inflammatory T cells. As control T cells were engineered with the angiomotin specific CAR #1061 of irrelevant specificity.

Anti-CD25 CAR #1035 T cells in increasing numbers (0.125-1×10⁴ cells per well) were co-incubated with CEA+LS174T cells (10⁴ cells per well) and anti-CEA CAR #700 T cells (0.125-1×10⁴ cells per well). After 48 hrs the viability of LS174T cells was determined. Anti-CEA CAR T cells eliminate CEA+LS174T cells dependent on the number of anti-CEA CAR T cells (FIG. 6). In the presence of anti-CD25 CAR T cells, the lytic activity of anti-CEA CAR T cells was reduced indicated by higher viability of LS174T cells. Reduction of the cytolytic reaction by anti-CD25 CAR T cells was specific since anti-angiomotin CAR T cells did not alter the cytolytic activity of anti-CEA CAR T cells. Data indicate that anti-CD25 CAR T cells have the capacity to reduce the inflammatory capacity of an antigen-specific immune response.

### Example 6

### T-Cells Having an Anti-CD25 CAR Eliminate Inflammatory CD25+ T-Cells and Prevent a Further Spread of Inflammation.

In this example, cytolytic T-cells were endowed with an anti-CD25 CAR, and the resultant elimination of CD25+-inflammatory cells was determined. Human T-cells were endowed with the following CARs by retroviral gene transfer as described further above:
#1035 [anti-hCD25-CD3ζ]
#1036 [anti-hCD25-CD28-CD3ζ]
#1037 [anti-hCD25-CD28dLck-CD3ζ]
#1576 [anti-hCD25/anti-CEA-CD28-CD3ζ]
#607 [anti-CEA-CD28-CD3ζ]
#2134 [anti-mCD25-mCD4TM-mCD28-mCD3ζ] (anti-mouse CD25)

The respective CAR-T-cells were co-cultivated with non-modified autologous T-cells in a ratio of 1:4, 1:8 and 1:16. For induction of an inflammatory T-cell-activation, the cells were incubated with IL-2 (500 U/ml), agonistic anti-CD3-antibody OKT3 (200 ng/ml) and the agonistic anti-CD28-antibody 15E8 (50 ng/ml). Under these conditions, CD25+ T-cells were induced. After 36 hours, the number of CD25+ T-cells in the presence/absence of the CAR T-cells was determined by flow cytometry. The respective results are shown in FIGS. 7A-7G and 8.

It turns out that the CAR-T-cells in accordance with the present invention having CAR #1035, #1036, #1037 are very effective in the elimination of CD25+ T-cells. The CAR #1037 having a deleted Lck binding site in the CD28-signal domain is slightly less active than the CAR without CD28-co-stimulation (#1035) or with non-modified CD28-co-stimulation (#1036). The bispecific CAR #1576 is less active than the monospecific CARs. As a comparison, CARs of irrelevant specificity (CEA, CAR #607) and a CAR having a specificity for mouse CD25 without cross reactivity for human CD25 were used. These show no significant suppression of CD25+ inflammatory T-cells.

### REFERENCES

Barth S, Huhn M, Wels W, Diehl V, Engert A. Construction and in vitro evaluation of RFTS(scFv)-ETA′, a new recombinant single-chain immunotoxin with specific cytotoxicity toward CD25+ Hodgkin-derived cell lines. Int J Mol Med 1998; 1:249-56.
Golumba-Nagy V, Kuehle J, Abken H. Genetic Modification of T Cells with Chimeric Antigen Receptors: A Laboratory Manual. Hum Gene Ther Methods 2017; 28:302-9. doi:10.1089/hgtb.2017.083.
Hombach A, Heuser C, Gerken M, Fischer B, Lewalter K, Diehl V, et al. T cell activation by recombinant FcepsilonRI gamma-chain immune receptors: an extracellular spacer domain impairs antigen-dependent T cell activation but not antigen recognition. Gene Ther 2000; 7:1067-75. doi:10.1038/sj.gt.3301195.
Hombach A, Wieczarkowiecz A, Marquardt T, Heuser C, Usai L, Pohl C, et al. Tumor-specific T cell activation by recombinant immunoreceptors: CD3 zeta signaling and CD28 costimulation are simultaneously required for efficient IL-2 secretion and can be integrated into one combined CD28/CD3 zeta signaling receptor molecule. J Immunol Baltim Md. 1950 2001; 167:6123-31.
Hombach A, Hombach A A, Abken H. Adoptive immunotherapy with genetically engineered T cells: modification of the IgG1 Fc “spacer” domain in the extracellular moiety of chimeric antigen receptors avoids “off-target” activation and unintended initiation of an innate immune response. Gene Ther 2010; 17:1206-13. doi:10.1038/gt.2010.91.
Hombach A A, Gorgens A, Chmielewski M, Murke F, Kimpel J, Giebel B, et al. Superior Therapeutic Index in Lymphoma Therapy: CD30(+) CD34(+) Hematopoietic Stem Cells Resist a Chimeric Antigen Receptor T-cell Attack. Mol Ther J Am Soc Gene Ther 2016; 24:1423-34. doi:10.1038/mt.2016.82.
Weijtens M E, Willemsen R A, Hart E H, Bolhuis R L. A retroviral vector system “STITCH” in combination with an optimized single chain antibody chimeric receptor gene structure allows efficient gene transduction and expression in human T lymphocytes. Gene Ther 1998; 5:1195-203. doi:10.1038/sj.gt.3300696.

1. A protein comprising:
(i) a CD25-specific binding domain,
(ii) a linker domain, connecting domain (i) and domain (iii),
(iii) a transmembrane domain, and
(iv) a signalling domain,
wherein the signalling domain comprises
a primary human signal chain, and
an intracellular co-stimulatory signalling chain(s),
or
a fusion of said intracellular co-stimulatory signal chain(s) with the intracellular domain of a primary human signal chain; and,
optionally, further comprising a secretion signal peptide.

2. The protein of claim 1, wherein the signalling domain (iv) is derived from
(1) the human CD3 zeta chain,
(2) the intracellular domain of human CD28 linked to the intracellular domain of human CD3 zeta chain, or
(3) the intracellular domain of human CD28 linked to the intracellular domain of human CD3 zeta chain, wherein CD28 comprises at least one mutation.

3. The protein of claim 1, wherein the CD25-specific binding domain (i) is an anti-CD25 single chain Fv (scFv) fragment,
or wherein the CD25-specific binding domain (i) is derived from or comprises IL-2.

4. The protein according to claim 1, wherein the linker domain (ii) is human IgG1 Fc domain or a domain derived therefrom.

5. The protein according to claim 1, further comprising an N-terminal secretion signal peptide, preferably immunoglobulin heavy chain signal peptide, or immunoglobulin light chain signal peptide, such as the IgG kappa light chain leader sequence.

6. The protein according to claim 1, comprising an amino acid sequence selected from SEQ ID NOs: 4 to 6,
or an amino acid sequence that has at least 85% sequence identity to an amino acid sequence of SEQ ID NOs: 4 to 6, or wherein said protein consists of an amino acid sequence selected from SEQ ID NOs: 4-6.

7. A nucleic acid encoding the protein of claim 1.

8. The nucleic acid of claim 7, comprising the nucleic acid encoding the amino acid sequence of SEQ ID NO: 1 or
comprising the nucleic acid sequence of SEQ ID NO: 7 or their complementary sequences or sequences that have at least 85% sequence identity, and/or comprising the nucleic acid encoding for the amino acid sequence of SEQ ID NOs: 2 or 3,
comprising the nucleic acid sequence of SEQ ID NOs: 8 or 9, or their complementary sequences or sequences that have at least 85% sequence identity.

9. The nucleic acid of claim 7, comprising the nucleic acid encoding an amino acid sequence selected from SEQ ID NOs: 4 to 6 or a nucleic acid sequence selected from SEQ ID NOs: 10 to 12 or their complementary sequences or sequences that have at least 85%, sequence identity.

10. An expression construct for expressing the protein of claim 1 in a cell.

11. A host cell expressing a protein of claim 1 or comprising a nucleic acid encoding a protein of claim 1, or comprising an expression construct for expressing a protein of claim 1.

12. A method for generating CD25-specific immune cells, wherein said method comprises use of a protein of claim 1, a nucleic acid encoding a protein of claim 1 or an expression construct for expressing a protein of claim 1.

13. A pharmaceutical composition, comprising:
(i) at least one protein of claim 1, at least one nucleic acid encoding a protein of claim 1, at least one expression construct for expressing a protein of claim 1, or at least one host cell expressing a protein of claim 1, and
(ii) optionally, a pharmaceutically acceptable excipient and/or carrier.

14-15. (canceled)

16. A method for treating inflammation, wherein said method comprises administering, to a subject in need of such treatment, a pharmaceutical composition of claim 13.

17. The method of claim 16, wherein the inflammation comprises chronic inflammation associated with multiple sclerosis, inflammatory bowel disease, Crohn's disease, ulcerative colitis, type-1 diabetes, psoriasis, rheumatoid arthritis, systemic lupus erytematosus, Hashimoto's thyreoiditis, Addison's disease, Graves' disease, Sjögren's syndrome, myastenia gravis, auto-immune vasculitis, pernicious anemia, or celiac disease.

18. The protein of claim 1, wherein the CD25-specific binding domain comprises an anti-CD25 single chain Fv (scFv) fragment; the linker domain comprises a human immunoglobulin Fc domain; the signalling domain comprises a primary human signal domain derived from human CD3 zeta chain or a human FcεRI gamma chain, and an intracellular co-stimulatory signally chain derived from human CD28, 4-1BB, OX40 or CD27.

19. The protein of claim 2, wherein CD28 comprises a deletion of the Lck binding site.

20. The protein of claim 3, wherein the CD25-specific binding domain (i) is an anti-CD25 single chain Fv (scFv) fragment, comprising the amino acid sequence of SEQ ID NO: 1.

21. The protein of claim 4, wherein the linker domain (ii) is human IgG1 Fc domain or a domain derived therefrom, comprising the amino acid sequence of SEQ ID NOs: 2 or 3.

22. The host cell of claim 11, wherein the host cell is selected from T cells and regulatory T (Treg) cells.


SEQUENCE LISTING (16 sequences)

SEQ ID NO: 1 - protein, 239 aa, Artificial Sequence
  anti-CD25 scFv
QVKLQQSGTVLARPGASVKMSCKASGYRFTNYWMHWVKQRPGQGLEWIGVIYPGNSDTSYNQKFKGKAKLTAVTSASTAYMELSSLTNEDSAVYYCTREGEGSDYWGQGTTVTVSSGGGGSGGGGSGGGGSQIVLTQSPATMAASPGEKITITCSASSSISSNYLHWYQQKPGFSPKLLIYRTSNLASGVPARFSGSGSGTSYSLTIGTMEAEDVATYYCQQGSSIPYTFGGGTKLELK

SEQ ID NO: 2 - protein, 236 aa, Artificial Sequence
  linker domain human IgG1 Fc
DPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKK

SEQ ID NO: 3 - protein, 234 aa, Artificial Sequence
  linker domain human IgG1 delta Fc
PAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKK

SEQ ID NO: 4 - protein, 611 aa, Artificial Sequence
  CAR # 1035
QVKLQQSGTVLARPGASVKMSCKASGYRFTNYWMHWVKQRPGQGLEWIGVIYPGNSDTSYNQKFKGKAKLTAVTSASTAYMELSSLTNEDSAVYYCTREGEGSDYWGQGTTVTVSSGGGGSGGGGSGGGGSQIVLTQSPATMAASPGEKITITCSASSSISSNYLHWYQQKPGFSPKLLIYRTSNLASGVPARFSGSGSGTSYSLTIGTMEAEDVATYYCQQGSSIPYTFGGGTKLELKDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 659 aa, Artificial Sequence
  CAR # 1036
QVKLQQSGTVLARPGASVKMSCKASGYRFTNYWMHWVKQRPGQGLEWIGVIYPGNSDTSYNQKFKGKAKLTAVTSASTAYMELSSLTNEDSAVYYCTREGEGSDYWGQGTTVTVSSGGGGSGGGGSGGGGSQIVLTQSPATMAASPGEKITITCSASSSISSNYLHWYQQKPGFSPKLLIYRTSNLASGVPARFSGSGSGTSYSLTIGTMEAEDVATYYCQQGSSIPYTFGGGTKLELKDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 659 aa, Artificial Sequence
  CAR # 1037
QVKLQQSGTVLARPGASVKMSCKASGYRFTNYWMHWVKQRPGQGLEWIGVIYPGNSDTSYNQKFKGKAKLTAVTSASTAYMELSSLTNEDSAVYYCTREGEGSDYWGQGTTVTVSSGGGGSGGGGSGGGGSQIVLTQSPATMAASPGEKITITCSASSSISSNYLHWYQQKPGFSPKLLIYRTSNLASGVPARFSGSGSGTSYSLTIGTMEAEDVATYYCQQGSSIPYTFGGGTKLELKDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQAYAAARDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 717 bp, Artificial Sequence
  anti-CD25 scFv
caggtgaagctgcagcagtctgggactgtgctggcaaggcctggggcttccgtgaagatgtcctgcaaggcttctggctacaggtttaccaactactggatgcactgggtaaaacagaggcctggacagggtctagaatggattggtgttatttatcctggaaatagtgatactagctacaaccagaagttcaagggcaaggccaaactgactgcagtcacatccgccagcactgcctacatggagctcagcagcctgacaaatgaggactctgcggtctattactgtacaagagagggagaaggctctgactactggggccaagggaccacggtcaccgtctcctcaggtggaggcggttcaggcggaggtggctctggcggtggcggatcgcaaattgttctcacccagtctccagcaaccatggctgcatctcccggggagaagatcactatcacctgcagtgccagctcaagtataagttccaattacttgcattggtatcagcagaagccaggattctcccctaaactcttgatttataggacttccaatctggcttctggagtcccagctcgcttcagtggcagtgggtctgggacctcttactctctcacaattggcaccatggaggctgaagatgttgccacttactactgccagcagggtagtagtataccgtacacgttcggaggggggaccaagctggagctgaag

SEQ ID NO: 8 - DNA, 708 bp, Artificial Sequence
  linker domain human IgG1 Fc
gatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaa

SEQ ID NO: 9 - DNA, 702 bp, Artificial Sequence
  linker domain human IgG1 delta Fc
cccgccgagcccaaatctcctgacaaaactcacacatgcccaccgtgcccagcacctccagtcgcgggaccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaa

SEQ ID NO: 10 - DNA, 1899 bp, Artificial Sequence
  CAR # 1035, including leader sequence
atggattttcaggtgcagattttcagcttcctgctaatcagtgcctcagtcataatgtctagacaggtgaagctgcagcagtctgggactgtgctggcaaggcctggggcttccgtgaagatgtcctgcaaggcttctggctacaggtttaccaactactggatgcactgggtaaaacagaggcctggacagggtctagaatggattggtgttatttatcctggaaatagtgatactagctacaaccagaagttcaagggcaaggccaaactgactgcagtcacatccgccagcactgcctacatggagctcagcagcctgacaaatgaggactctgcggtctattactgtacaagagagggagaaggctctgactactggggccaagggaccacggtcaccgtctcctcaggtggaggcggttcaggcggaggtggctctggcggtggcggatcgcaaattgttctcacccagtctccagcaaccatggctgcatctcccggggagaagatcactatcacctgcagtgccagctcaagtataagttccaattacttgcattggtatcagcagaagccaggattctcccctaaactcttgatttataggacttccaatctggcttctggagtcccagctcgcttcagtggcagtgggtctgggacctcttactctctcacaattggcaccatggaggctgaagatgttgccacttactactgccagcagggtagtagtataccgtacacgttcggaggggggaccaagctggagctgaaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 11 - DNA, 2043 bp, Artificial Sequence
  CAR # 1036, including leader sequence
atggattttcaggtgcagattttcagcttcctgctaatcagtgcctcagtcataatgtctagacaggtgaagctgcagcagtctgggactgtgctggcaaggcctggggcttccgtgaagatgtcctgcaaggcttctggctacaggtttaccaactactggatgcactgggtaaaacagaggcctggacagggtctagaatggattggtgttatttatcctggaaatagtgatactagctacaaccagaagttcaagggcaaggccaaactgactgcagtcacatccgccagcactgcctacatggagctcagcagcctgacaaatgaggactctgcggtctattactgtacaagagagggagaaggctctgactactggggccaagggaccacggtcaccgtctcctcaggtggaggcggttcaggcggaggtggctctggcggtggcggatcgcaaattgttctcacccagtctccagcaaccatggctgcatctcccggggagaagatcactatcacctgcagtgccagctcaagtataagttccaattacttgcattggtatcagcagaagccaggattctcccctaaactcttgatttataggacttccaatctggcttctggagtcccagctcgcttcagtggcagtgggtctgggacctcttactctctcacaattggcaccatggaggctgaagatgttgccacttactactgccagcagggtagtagtataccgtacacgttcggaggggggaccaagctggagctgaaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgcccccccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 12 - DNA, 2043 bp, Artificial Sequence
  CAR # 1037, including leader sequence
atggattttcaggtgcagattttcagcttcctgctaatcagtgcctcagtcataatgtctagacaggtgaagctgcagcagtctgggactgtgctggcaaggcctggggcttccgtgaagatgtcctgcaaggcttctggctacaggtttaccaactactggatgcactgggtaaaacagaggcctggacagggtctagaatggattggtgttatttatcctggaaatagtgatactagctacaaccagaagttcaagggcaaggccaaactgactgcagtcacatccgccagcactgcctacatggagctcagcagcctgacaaatgaggactctgcggtctattactgtacaagagagggagaaggctctgactactggggccaagggaccacggtcaccgtctcctcaggtggaggcggttcaggcggaggtggctctggcggtggcggatcgcaaattgttctcacccagtctccagcaaccatggctgcatctcccggggagaagatcactatcacctgcagtgccagctcaagtataagttccaattacttgcattggtatcagcagaagccaggattctcccctaaactcttgatttataggacttccaatctggcttctggagtcccagctcgcttcagtggcagtgggtctgggacctcttactctctcacaattggcaccatggaggctgaagatgttgccacttactactgccagcagggtagtagtataccgtacacgttcggaggggggaccaagctggagctgaaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccaggcctatgccgccgcacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 13 - protein, 41 aa, Artificial Sequence
  CD28 delta Lck intracellular domain
RSKRSRLLHSDYMNMTPRRPGPTRKHYQAYAAARDFAAYRS

SEQ ID NO: 14 - DNA, 123 bp, Artificial Sequence
  encoding SEQ ID NO. 13
aggagtaagaggagcaggctcctgcacagtgactacatgaacatgactccccgccgccccgggcccacccgcaagcattaccaggcctatgccgccgcacgcgacttcgcagcctatcgctcc

SEQ ID NO: 15 - protein, 21 aa, Artificial Sequence
  IgG kappa light chain leader sequence
MDFQVQIFSFLLISASVIMSR

SEQ ID NO: 16 - DNA, 63 bp, Artificial Sequence
  example of nucleotide sequence encoding SEQ ID NO. 15
atggattttcaggtgcagattttcagcttcctgctaatcagtgcctcagtcataatgtctaga